(19) United States
(12) Patent Application Publication
(10) Pub. No.: US 2024/0299664 A1
(43) Pub. Date: Sep. 12, 2024

(54) AUTO-INJECTOR HOUSING LATCH

(71) Applicant: Owen Mumford Limited, Woodstock (GB)

(72) Inventor: Matthew David FARMER, Woodstock, Oxfordshire (GB)

(21) Appl. No.: 18/547,583

(22) PCT Filed: Feb. 6, 2022
(86) PCT No.: PCT/EP2022/052800

§ 371 Date: Aug. 23, 2023

(30) Foreign Application Priority Data
Feb. 28, 2021 (GB) 2102848.5

(51) Int. Cl. A61M 5/20; A61M 5/315; A61M 5/32

(52) U.S. Cl. CPC A61M 5/2033; A61M 5/3158; A61M 5/3204

(57) ABSTRACT

An auto-injector for receiving and operating a syringe to facilitate injection of medicament into a subject, the auto-injector comprises an openable lid to allow insertion and removal of the syringe, a lid latch operable to latch the lid in a closed position, and a needle safety shroud moveable between a first retracted position and a second extended position. The auto-injector is configured such that, when the safety shroud is in the first position, the lid latch cannot be released preventing the lid from being opened and, when the safety shroud is in the second position, the lid latch can be released allowing the lid to be opened.

### TECHNICAL FIELD

The invention relates to auto-injectors for use with syringes. The invention may relate to, but need not be limited to, auto-injectors for use with syringes having a safety shroud.

### BACKGROUND

Auto-injectors are devices for receiving and operating syringes automatically on activation by a user. Auto-injectors typically include a plunger driver that engages with a plunger of a syringe inserted into the auto-injector and one or more drive springs that are arranged to couple to and apply a force to the plunger of the syringe. To inject a drug, the user holds the auto-injector against the injection site and triggers the auto-injector, causing the plunger driver to drive the plunger of the syringe forward. This firstly pushes the entire syringe through the injector housing resulting in needle insertion. The syringe body then bottoms out in the housing such that continued force on the plunger results in the plunger travelling into the drug-filled barrel of the syringe. The drug is thereby forced through the needle into the injection site.

Some auto-injectors include a safety shroud that is deployed after the injection is complete. The safety shroud covers the needle as the auto-injector is removed from the injection site to prevent accidental needle stabbing during post-injection handling of the auto-injector. However, if the auto-injector is of a reusable type, the user still needs to remove the syringe from the auto-injector thereby exposing the needle and increasing the risk of injury. To minimise this risk, the syringe itself instead of or in addition to the auto-injector may be provided with a safety shroud. In such cases, the auto-injector is provided with a mechanism to deploy the safety shroud of the syringe.

Syringes with their own safety shrouds may be broadly split into ‘active’ and ‘passive’ syringes. Active syringes with a safety shroud typically require some action by a user to deploy the safety shroud, for example the pressing of a trigger button. Such action may be taken during or after removal of the needle from the injection site. Passive syringes with a safety shroud on the other hand deploy the safety shroud without any specific action by the user beyond that which is usually taken to use the syringe. For example, movement of a plunger beyond a certain point during injection of a drug may trigger the release of a spring-loaded safety shroud or, in the case of an auto-injector, the auto-injector plunger driver may bias the safety shroud towards its closed position.

To load a syringe into an auto-injector, the auto-injector may be provided with a lid, a door, a hatch or the like. For example, a side of a housing of the auto-injector may be mounted on a hinge, allowing the side of the auto-injector to pivot open, providing access to the inside of the auto-injector to load the syringe. Alternatively, the housing of the auto-injector may comprise a separable rear part that can be disconnected to provide access to load the syringe. The lid may be kept closed with a latch. To open the lid the user is typically required to operate latch, for example, by pressing a button, to release it.

In some cases, the closing movement of the lid may be used to prime the drive spring(s) of the auto-injector. For example, the closing movement of the lid compresses the auto-injector drive spring(s). When the lid is fully closed, the drive spring(s) is fully primed and the latch of the lid prevents the lid from springing open until the user operates the latch to release it. If the operates the latch before the injection is complete, the force of the drive spring primed by the closing movement of the lid may now act in the opposite direction to the priming movement causing the lid to spring open prematurely, before the injection is complete.

An exemplary auto-injector is proposed in WO/1170/064927A1.

### SUMMARY

According to a first aspect, there is provided, an auto-injector for receiving and operating a syringe to facilitate injection of medicament into a subject, the auto-injector comprises an openable lid to allow insertion and removal of the syringe, a lid latch operable to latch the lid in a closed position, and a needle safety shroud moveable between a first retracted position and a second extended position. The auto-injector is configured such that, when the safety shroud is in the first position, the lid latch cannot be released preventing the lid from being opened and, when the safety shroud is in the second position, the lid latch can be released allowing the lid to be opened.

Optionally, the auto-injector comprises: a plunger driver configured on activation of the auto-injector to drive a plunger of the syringe received in the auto-injector forward within the auto-injector to operate the syringe; and a housing for receiving the syringe, the housing comprising a channel for receiving the safety shroud.

Optionally, the lid latch comprises: a button operable to release the lid latch, the button comprising a shroud engagement member moveable on operation of the button from a position outside of the channel to a position inside the channel; wherein the safety shroud in the first retracted position blocks the shroud engagement member from moving into the channel to prevent operation of the button; and wherein the safety shroud in the second extended position allows movement of the shroud engagement member into the channel to allow operation of the button.

Optionally, the safety shroud comprises a blocking surface configured to engage the shroud engagement member when the safety shroud is in the first retracted position to block movement of the shroud engagement member into the channel.

Optionally, the lid comprises a hooked engagement member for engaging the button to latch the lid in the closed position; wherein the auto-injector is configured such that operation of the button releases the hooked engagement member to allow the lid to open.

Optionally, the housing comprises a pivot configured to mount the button, the button being moveable about the pivot to release the hooked engagement member on operation of the button.

Optionally, the auto-injector comprises a biasing member configured to bias the shroud engagement member away from the channel.

Optionally, responsive to the subject pressing a forward portion of the safety shroud onto an injection site, the safety shroud is configured to move to the first retracted position.

Optionally, responsive to the subject moving the safety shroud from the injection site, the safety shroud is configured to move to the second extended position.

Optionally, the safety shroud is configured to move to the second extended position responsive to a continuous force applied by the plunger driver.

Optionally, plunger driver is configured to lock out the safety shroud in the second extended position.

Optionally, the lid is configured to hinge between an open position and a closed position.

Optionally, the lid is configured on a closing movement thereof to prime a drive spring of the plunger driver.

According to a second aspect, there is provided an auto-injector according to the aspect described above in combination with a syringe comprising a syringe safety shroud configured to cover a needle of the syringe when in a closed position.

Optionally, the syringe safety shroud is configured to lock out in the closed position responsive to a continuous force applied by the plunger driver.

According to a third aspect, there is provided a kit of parts comprising: the auto-injector according to the aspect described above; and a safety syringe comprising a syringe safety shroud configured to cover a needle of the syringe when in a closed position.

Optionally, the syringe safety shroud is configured to lock out in the closed position responsive to a continuous force applied by the plunger driver.

### BRIEF DESCRIPTION OF THE DRAWINGS

Exemplary embodiments are disclosed herein with reference to the accompanying drawings, in which:

FIG. 1a shows a view of an auto-injector.

FIG. 1b shows a view of the auto-injector of FIG. 1b with a safety syringe received therein.

FIG. 1c shows a cut away view of the auto-injector of FIG. 1b with partially closed lid.

FIG. 1d shows a cut away view of the auto-injector of FIG. 1b with the lid in a closed position.

FIG. 2a shows a plan view of a forward part of the auto-injector of FIG. 1d with a lid release in an unlocked position.

FIG. 2b shows a plan view of the forward part of the auto-injector of FIG. 1d with a lid release in a locked position.

### DETAILED DESCRIPTION

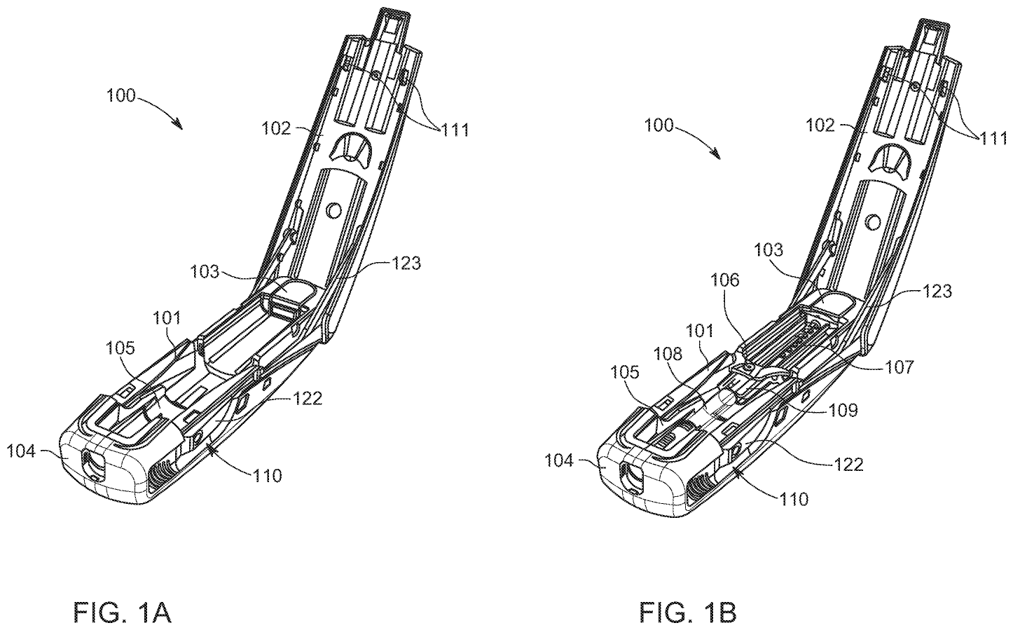

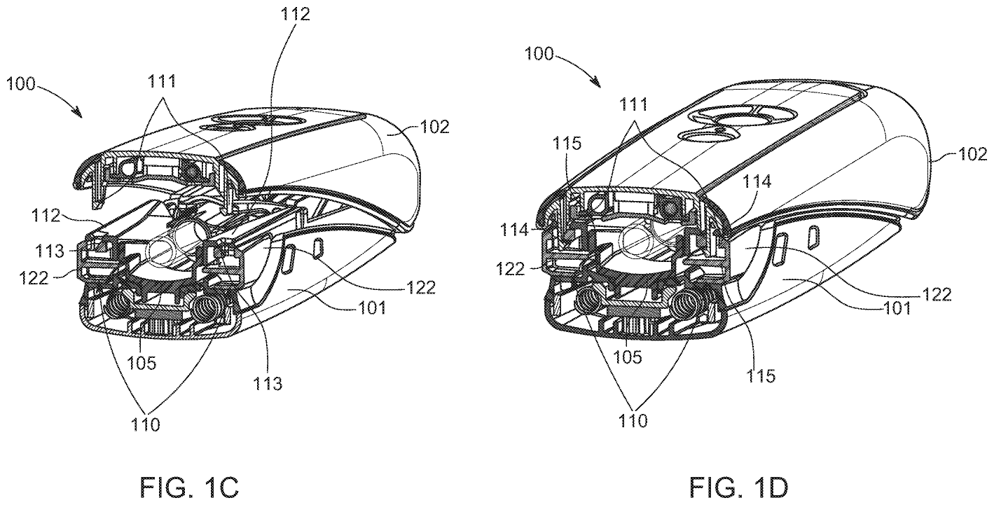

FIG. 1a shows a view of an auto-injector 100 for receiving and operating a syringe to facilitate injection of medicament into a subject. The auto-injector comprises a housing 101 having an openable lid 102, a plunger driver 103 comprising a pair of drive springs (not shown), a removable cap 104, and a needle safety shroud 105. The lid 102 is configured to open to allow insertion and removal of a syringe, for example a safety syringe, in the auto-injector 100 prior to use. The plunger driver 102 is configured to couple to a plunger of the syringe inserted in the auto-injector 100, for example by engaging a head of the plunger at its rear surface. The removable cap 104 covers a forward end of the auto-injector 100 prior to use. The safety shroud 105 is configured to move forwards and rearwards relative to the housing 101, for example responsive to a user pressing and releasing a forward portion of the safety shroud 105 extending from a forward end of the auto-injector 100 onto an injection site. The auto-injector 100 further comprises a lid latch 110. The lid latch 110 is operable to latch the lid 102 in a closed position. The lid 102 comprises two hooked engagement members 111 configured for engagement with the lid latch 110. The lid latch 110 comprises a button 122 operable to release the lid latch 110.

FIG. 1b shows a view of the auto-injector of FIG. 1a with a safety syringe 106 received therein. The safety syringe 106 comprises a plunger 107 configured, responsive to a force applied thereon by the plunger driver 103, to move forwards within the auto-injector 100 to force the contents of a barrel 108 of the safety syringe 106 through a needle (not shown) of the safety syringe 106 into an injection site. The safety syringe 106 further comprises a syringe safety shroud 109 configured to travel forwards over the barrel 108 coupled with the forward movement of the plunger, decouple from the plunger when the plunger reaches the end of the barrel 108 and continue further forward movement to cover a needle of the safety syringe 106 inside the housing 100 at the end of an injection to allow for safe handling of the safety syringe 106 after the injection is complete and after the safety syringe 106 is removed from the auto-injector 100.

FIG. 1c shows a cut away view of the auto-injector 100 of FIG. 1b with the lid partially closed. As the lid 102 pivots closed towards the rest of the housing 101, the hooked engagement members 111 move towards the lid latch 110.

FIG. 1d shows a cut away view of the auto-injector 100 of FIG. 1b with the lid fully closed. The hook-shaped portions 114 of the hooked engagement members 111 have moved through corresponding openings 112 in the housing 101 and corresponding openings 113 in the lid latch button 122, latching inside of the button 122. When the hooked engagement members 111 are latched to button 122, the lid 102 is unable to open and will thus remain closed.

To open the lid 102, the hooked portions 114 are unlatched from the inside of the button 122 by actuating the button 122 in an inwards direction towards a central axis of the auto-injector, thereby freeing the hooked portion 114 from its engagement with the button 122. The button 122 of the lid latch 110 is accordingly moveable on actuation by the user between a first position in which the lid 102 is latched to the button and unable to open and a second position allowing the lid 102 to open. To prevent accidental, premature unlatching of the hooked portions 114 from the button 122 due to inadvertent actuation of the button 122 inwards, the button 122 is provided with one or more shroud engagement members 115 configured for engagement with the safety shroud 105. As will be described in more detail below, blocking movement of the one or more shroud engagement members 115 using the safety shroud 105 prevents the button 122 from moving thereby rendering it inoperable and the lid latch 110 unable to be released until the safety shroud 105 moves out of the way of the one or more shroud engagement members 115.

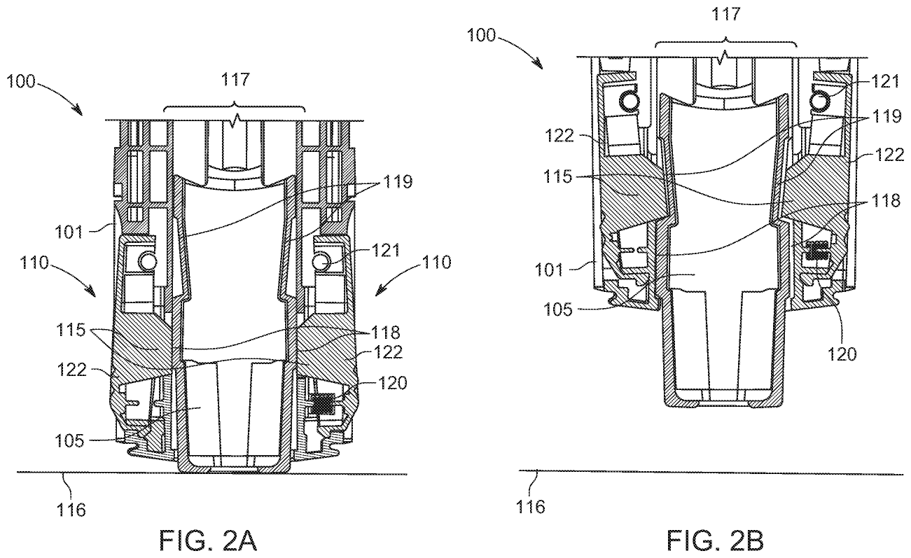

FIG. 2a shows an illustrative plan view of a forward part of the auto-injector 100 of FIGS. 1a-1d pressed onto an injection site 116. As described above, the auto-injector 100 comprises a housing 101 for receiving a syringe, a lid 102, and a lid latch 110. As also described above, the lid latch 110 comprises a button 122 that engages one or more hooked engagement members 111 (not shown) and latches them in place to prevent the lid 102 from opening. The auto-injector 100 may further comprise a biasing member 120 configured to bias the shroud engagement member 115 away from the channel 117, and the housing 101 may comprise one or more pivots 121 configured to mount the button 122 in the auto-injector 100 thereby enabling the button 122 to be moveable against the bias about the pivot 121 responsive to a user actuating the button 122.

The housing 101 defines a channel 117 for receiving the safety shroud 105. The safety shroud 105 is configured to move forwards and rearwards in the channel 117 responsive to a forward portion of the safety shroud 105 being pressed onto an injection site. Thus, when the safety shroud 105 is pressed onto an injection site by a user, the safety shroud 105 is moved to a first retracted position in the channel 117 as is shown in FIG. 2a. Conversely, when the safety shroud 105 is moved from the injection site, the safety shroud 105 moves to a second extended position.

The shroud engagement member 115 of the button 122 is moveable from a position outside of the channel 117 to a position inside the channel 117 on operation of the button 122. When the safety shroud 105 is in the first retracted position it blocks the shroud engagement member 115 from moving into the channel 117 thereby preventing operation of the button 122 and preventing release of the lid latch 110. Conversely, when the safety shroud 105 is in the second extended position it allows movement of the shroud engagement member 115 into the channel 117 thereby allowing operation of the button 122 and allowing release of the lid latch 110.

The safety shroud 105 thus comprises one or more blocking surfaces 118 configured to engage the shroud engagement member 115 when the safety shroud 105 is in the first retracted position thereby blocking movement of the shroud engagement member 115 into the channel 117. When the safety shroud 105 is in the second extended position, the blocking surfaces 118 are out of the way of the shroud engagement member which is then moveable, at least partially, laterally into and out of the channel 117 from a side of the channel 117. By blocking movement of the shroud engagement member 115 into the channel 117, the button 122 is made inoperable as it is prevented from moving is thus kept in a locked position until the safety shroud 105 has moved from the first retracted position in the channel 117 to the second extended position. With the button 122 of the lid latch 110 kept in its locked position, the hooked engagement members 111 of the lid 102 cannot disengage from button 122 and the lid 102 is thereby locked closed while the safety shroud 105 is in its first retracted position. Thus, when the safety shroud 105 is pressed against an injection site during an injection causing the safety shroud 105 to move to its first retracted position, the lid 102 of the auto-injector 100 cannot accidentally be opened by a user.

Once an injection is complete, the safety shroud 105 may be lifted from the injection site 116 allowing the safety shroud 105 to move forwards in the channel 117 to the second extended position, for example under a force applied by a drive spring of the auto-injector 100.

FIG. 2b shows a cross-sectional view of the forward part of the auto-injector 100 lifted away from the injection site 116 with the safety shroud 105 having moved from the first retracted position into the second extended position in the channel 117.

In the second extended position, the one or more blocking surfaces 118 have disengaged from and moved out of the way of the one or more shroud engagement members 115 allowing them to move laterally inwards responsive to a user actuating the button 122 of the lid latch 110, for example by pressing it inwards. The safety shroud 105 may comprise one more receiving surfaces 119 configured to engage with the one or more shroud engagement members 115 once inside the channel 117. With the button 122 actuated, the hooked portion 114 of the hooked engagement members 111 disengage from the button 122 allowing the lid 102 to open. With the lid 102 open, a user may safely remove the syringe from the auto-injector 100.

When the safety shroud 105 is in the second extended position, a forward portion of the safety shroud 105 covers the needle of the syringe to prevent accidental needle stab injuries. The safety shroud 105 may further be locked out into this second, extended position covering the needle for example responsive to a continuous force applied by the plunger driver.

As used herein, the term forward and rearward refer respectively to a needle end of an auto-injector that is pressed against the user's skin and the end opposite the needle end. Forward and rearward movement thus refer to movement towards the needle end or movement away from the needle end of the auto-injector. The term forward stroke thus refers to the total forward movement of a component of the auto-injector from a starting point towards the needle end to an endpoint. For example, the forward stroke of the plunger driver thus refers to the movement towards the needle end required to move from a starting to an endpoint. The endpoints of the forward stroke of the syringe plunger and the plunger driver may or may not coincide.

The skilled person will envisage further embodiments of the invention without departing from the scope of the appended claims.

For example, in addition to the locking out of the safety shroud of the auto-injector, the safety shroud of the safety syringe may be configured to lock out in a closed position configured to cover a needle of the syringe responsive to a continuous force applied by the plunger driver as part of the forward stroke of the plunger driver.

Further, the lid latch may additionally comprise multiple buttons or other actuation surfaces, for example arranged symmetrically on either side of the auto-injector. The buttons may be actuatable through one or more corresponding openings in the housing of the auto-injector.

Further, the plunger driver may comprise one or more drive springs that may be compression, tension, and or torsion springs. These may be arranged rearwards and aligned in the same axis as the syringe received in the auto-injector. Alternatively, the one or more drive springs may be arranged under, above or adjacent to a plane of the syringe thereby allowing the total length of the device to be reduced compared to devices where the one or more drive springs are arranged rearwards in the same axis as the syringe.

Further, the lid may be configured to prime the drive springs on an opening and/or closing movement thereof. This may be achieved by, for example, coupling the lid to the drive springs, for example through the plunger driver, with one or more linkages 123. Movement of the lid in the opening or closing direction compresses or tensions the drive springs through the linkages 123 to prime the drive springs. For example, the linkages may be slidably coupled to the lid at one end and to the plunge driver at the other. As the lid moves, the linkages 123 slide in a channel In the lid thereby converting the movement of the lid as it hinges closed into a linear movement in the drive spring thereby compressing it or tensioning it depending on if the drive spring is a compression or a tension spring.

Further, the auto-injector may be provided with electronics and a means to visually or audibly indicate to a user how long the user must hold the auto-injector in position on the injection site at the end of the injection and to provide a countdown to ensure the user holds the auto-injector on the injection site for required time. The visual and/or audible indicator may comprise one or more light emitting diodes and/or a speaker configured to emit a sound.

1. An auto-injector for receiving and operating a syringe to facilitate injection of medicament into a subject, the auto-injector comprising an openable lid to allow insertion and removal of the syringe, a lid latch operable to latch the lid in a closed position, and a needle safety shroud moveable between a first retracted position and a second extended position, the auto-injector being configured such that, when the safety shroud is in the first position, the lid latch cannot be released preventing the lid from being opened and, when the safety shroud is in the second position, the lid latch can be released allowing the lid to be opened.

2. The auto-injector according to claim 1 comprising:
a plunger driver configured on activation of the auto-injector to drive a plunger of the syringe received in the auto-injector forward within the auto-injector to operate the syringe; and
a housing for receiving the syringe, the housing comprising a channel for receiving the safety shroud.

3. The auto-injector according to claim 2, wherein the lid latch comprises:
a button operable to release the lid latch, the button comprising a shroud engagement member moveable from a position outside of the channel to a position inside the channel on operation of the button;
wherein the safety shroud in the first retracted position blocks the shroud engagement member from moving into the channel to prevent operation of the button; and
wherein the safety shroud in the second extended position allows movement of the shroud engagement member into the channel to allow operation of the button.

4. The auto-injector according to claim 3,
wherein the safety shroud comprises a blocking surface configured to engage the shroud engagement member when the safety shroud is in the first retracted position to block movement of the shroud engagement member into the channel.

5. The auto-injector according to claim 1,
wherein the lid comprises a hooked engagement member for engaging the button to latch the lid in the closed position;
wherein the auto-injector is configured such that operation of the button releases the hooked engagement member to allow the lid to open.

6. The auto-injector according to claim 4, wherein the housing comprises a pivot configured to mount the button, the button being moveable about the pivot to release the hooked engagement member on operation of the button.

7. The auto-injector according to claim 1, comprising a biasing member configured to bias the shroud engagement member away from the channel.

8. The auto-injector according to claim 2, wherein responsive to the subject pressing a forward portion of the safety shroud onto an injection site, the safety shroud is configured to move to the first retracted position.

9. The auto-injector according to claim 8, wherein responsive to the subject moving the safety shroud from the injection site, the safety shroud is configured to move to the second extended position.

10. The auto-injector according to claim 9, wherein the safety shroud is configured to move to the second extended position responsive to a continuous force applied by the plunger driver.

11. The auto-injector according to claim 10, wherein the plunger driver is configured to lock out the safety shroud in the second extended position.

12. The auto-injector according to claim 1, wherein the lid is configured to hinge between an open position and a closed position.

13. The auto-injector according to claim 1, wherein the lid is configured on a closing movement thereof to prime a drive spring of the plunger driver.

14. The auto-injector according to claim 1 in combination with a syringe comprising a syringe safety shroud configured to cover a needle of the syringe when in a closed position.

15. The auto-injector according to claim 14, wherein the syringe safety shroud is configured to lock out in the closed position responsive to a continuous force applied by the plunger driver.

16. A kit of parts comprising:
the auto-injector according to claim 1; and
a safety syringe comprising a syringe safety shroud configured to cover a needle of the syringe when in a closed position.

17. The kit of parts according to claim 16, wherein the syringe safety shroud is configured to lock out in the closed position responsive to a continuous force applied by the plunger driver.
